(19) United States
(12) Patent Application Publication
(10) Pub. No.: US 2025/0114235 A1
(43) Pub. Date: Apr. 10, 2025

(54) NASAL STRIP FOR TREATING SLEEP APNEA

(71) Applicant: Skyler P. Stoner, Edmond, OK (US)

(72) Inventor: Skyler P. Stoner, Edmond, OK (US)

(21) Appl. No.: 18/909,478

(22) Filed: Oct. 8, 2024

Related U.S. Application Data
(60) Provisional application No. 63/588,840, filed on Oct. 9, 2023.

(51) Int. Cl. A61F 5/56

(52) U.S. Cl. CPC A61F 5/56

(57) ABSTRACT

Nasal strips for treating obstructive sleep apnea (OSA) and methods of use are herein disclosed, including a nasal strip comprising a substrate and an adhesive layer. The substrate is formed of a flexible, elastic material and has a bridge portion and first and second lateral portions extending from a side of the bridge portion. The bridge portion is sized and dimensioned to extend between first and second nasal sidewalls and across a bridge of a user's nose and to be positionable over at least a portion of an infratrochlear nerve of the user's nose. The first lateral portion is sized and dimensioned to be positionable over a portion of an anterior ethmoidal nerve on the first nasal sidewall of the user's nose. The second lateral portion is sized and dimensioned to be positionable over a portion of an anterior ethmoidal nerve on the second nasal sidewall of the user's nose.

### CROSS-REFERENCE TO RELATED APPLICATIONS

This application claims priority to the provisional patent application identified by U.S. Ser. No. 63/588,840, filed Oct. 9, 2023, the entire content of which is hereby expressly incorporated herein by reference.

### BACKGROUND

Sleep apnea is a group of common sleep disorders characterized by repeated interruptions in an affected person's breathing during sleep. Obstructive sleep apnea (OSA) is the most common form of sleep apnea. It is a condition in which the OSA-affected person's breathing is repeatedly interrupted during sleep due to physical blockage of the airway. These interruptions in breathing, known as apneas, result from the relaxation and collapse of throat muscles and tissues, leading to an airway obstruction. Apneas may persist for seconds to minutes and recur multiple times throughout the night. This fragmented sleep pattern impedes the OSA-affected person from getting restorative deep sleep, resulting in daytime fatigue, drowsiness, and difficulty staying awake during activities such as driving or working.

OSA has been demonstrated to impair cognitive functions, including but not limited to memory, concentration, and decision-making capabilities. Such impairments can adversely affect work performance, academic achievement, and daily functioning. Furthermore, OSA is also associated with significant health risks, particularly to the cardiovascular system. Cyclical reductions in blood oxygen levels and the resultant strain on the cardiovascular system have been correlated with an increased risk of high blood pressure, cardiac disease, stroke, and irregular heartbeat. Additionally, untreated OSA has been linked to metabolic conditions such as insulin resistance and type 2 diabetes. OSA may also contribute to weight gain through disruption of appetite-regulating hormones.

Moreover, auditory disturbances associated with OSA, such as loud snoring and episodes of choking or gasping, can negatively impact the sleep quality of bed partners of OSA-affected persons, potentially causing relationship strain and sleep disturbance for both affected individuals. Thus, OSA presents complex challenges that extend beyond nighttime interruptions, encompassing overall health, daily functioning, and interpersonal relationships.

Several limitations and disadvantages characterize current treatment modalities for OSA. One widely employed treatment involves continuous positive airway pressure (CPAP) therapy, which may be perceived as uncomfortable and cumbersome by many patients. The need to wear a mask and the associated noise generated by the CPAP device can disrupt sleep and present challenges for patient compliance and adaptation to the treatment regimen.

Surgical interventions, which may be considered for certain severe cases of OSA, carry inherent risks of complications, including but not limited to infection, bleeding, and scarring. Furthermore, the efficacy of surgical treatments may diminish over time, potentially necessitating additional procedures.

Another treatment modality for OSA includes the application of nasal strips incorporating dilators that exert a lifting force to expand the nasal passages. However, such nasal strips primarily address nasal congestion and demonstrate limited efficacy in treating OSA caused by anatomical issues with the throat.

### SUMMARY OF THE INVENTIVE CONCEPTS

Thus, there is a desire for a new and improved device for effectively treating obstructive sleep apnea (OSA) while reducing the limitations and disadvantages characterizing current treatment modalities. It is to such devices and methods of using the same that the present disclosure is directed.

The problem of effectively treating OSA with current nasal strips that include dilators for opening the nasal passages is solved by a nasal strip for treating OSA and a method of use thereof described herein.

In one aspect, the present disclosure includes a nasal strip for treating OSA, comprising: a substrate formed of a flexible, elastic material and having a skin-facing surface and a non-skin-facing surface opposite the skin-facing surface, the substrate having: a bridge portion having a first end, a second end opposite the first end, a first side, a second side opposite the first side, and a longitudinal axis extending between the first end and the second end, the bridge portion being sized and dimensioned to extend between a first nasal sidewall of a user's nose and a second nasal sidewall of the user's nose opposite the first nasal sidewall and across a bridge of the user's nose and to be positionable over at least a portion of an infratrochlear nerve of the user's nose to provide an epidermal tractional force parallel to a surface of application on the user's nose and in a direction toward the bridge of the user's nose to stimulate the infratrochlear nerves of the user's nose; a first lateral portion extending from the second side of the bridge portion, the first lateral portion being sized and dimensioned to be positionable over a portion of an anterior ethmoidal nerve on the first nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the first nasal sidewall of the user's nose; a second lateral portion extending from the second side of the bridge portion, the second lateral portion being sized and dimensioned to be positionable over a portion of an anterior ethmoidal nerve on the second nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the second nasal sidewall of the user's nose; and an adhesive layer disposed on the skin-facing surface of the substrate, the adhesive layer being operable to attach the substrate to an epidermal surface of the user's nose.

In another aspect, the present disclosure includes a nasal strip for treating OSA, comprising: a substrate formed of a flexible, elastic material and having a skin-facing surface and a non-skin-facing surface opposite the skin-facing surface, the substrate having: a bridge portion having a first end, a second end opposite the first end, a first side, a second side opposite the first side, and a longitudinal axis extending between the first end and the second end, the bridge portion being sized and dimensioned to extend between a first nasal sidewall of a user's nose and a second nasal sidewall of the user's nose opposite the first nasal sidewall and across a bridge of the user's nose and to be positionable over at least a portion of an infratrochlear nerve of the user's nose to provide an epidermal tractional force parallel to a surface of application on the user's nose and in a direction toward the bridge of the user's nose to stimulate the infratrochlear nerves of the user's nose; a plurality of lateral portion pairs, each of the plurality of lateral portion pairs including a first lateral portion extending from the first side of the bridge portion and a second lateral portion extending from the second side of the bridge portion; and an adhesive layer disposed on the skin-facing surface of the substrate, the adhesive layer being operable to attach the substrate to an epidermal surface of the user's nose; wherein the first lateral portion of each of the plurality of lateral portion pairs is sized and dimensioned to be positionable over a portion of an anterior ethmoidal nerve on the first nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the first nasal sidewall of the user's nose; and wherein the second lateral portion of each of the plurality of lateral portion pairs is sized and dimensioned to be positionable over a portion of an anterior ethmoidal nerve on the second nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the second nasal sidewall of the user's nose.

The combined tractional force applied by the nasal strip maintains a gentle, concerted stimulation of the infratrochlear nerves and anterior ethmoidal nerves, which may enhance neuroconnectivity and sensation, potentially affecting and engaging the activation energy (EA) and sustainability of target impulses and reflexes, respectively. The resulting neuro-harmonic effects may raise and expand key issues in the oral cavity and greater pharynx, potentially providing improved histological rigidity and flexibility, enhanced histologic impulse conductivity, and optimized structural and/or functional histological interfaces in the greater otolaryngological area via neuro-harmonic connective invigorated innervation stimulation.

The nasal strip described herein may directly or indirectly influence the histologic and/or neural conductivity related to innate defense impulses and reflexes that may aid in preventing and ceasing OSA events. The mild epidermal tractional force applied by the nasal strip described herein may induce a calming sensation that could facilitate sleep onset and improve sleep stage motility and efficiency, possibly reducing snoring duration. In some implementations, the nasal strip described herein may function as a nervous stimulation unit that could enhance audio and/or vibratory feedback and audiovocal tonal harmony.

Users of the nasal strip described herein may experience a reduction in the activation energy required for physio-feedback-induced physical repositioning during sleep and for the mammalian reflect, potentially enabling more restful sleep. Additional benefits of the nasal strip described herein may include improved cardiopulmonary efficiency through reduced cardiopulmonary stress and blood pressure, specifically ocular pressure.

The details of one or more implementations of the subject matter described in this specification are set forth in the accompanying drawings and the description below.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a top plan view of an exemplary implementation of a nasal strip for treating obstructive sleep apnea (OSA) constructed in accordance with the inventive concepts disclosed herein.

FIG. 2A is a side elevational view of the nasal strip of FIG. 1.

FIG. 2B is a bottom plan view of another implementation of the nasal strip of FIGS. 1 and 2A.

FIG. 3 is a top plan view of another exemplary implementation of a nasal strip for treating OSA constructed in accordance with the inventive concepts disclosed herein.

FIG. 4A is a side elevational view of the nasal strip of FIG. 3.

FIG. 4B is a bottom plan view of another implementation of the nasal strip of FIGS. 3 and 4A.

FIG. 5 is a diagrammatic view of the nasal strip of FIGS. 1 and 2A applied to a user's nose, illustrating infratrochlear nerves and anterior ethmoidal nerves of the user's nose.

FIG. 6 is another diagrammatic view of the nasal strip of FIGS. 1 and 2A applied to the user's nose, illustrating an epidermal traction stimulation of the infratrochlear nerves and the anterior ethmoidal nerves of the user's nose.

FIG. 7 is a diagrammatic view of the nasal strip of FIGS. 3 and 4A applied to a user's nose, illustrating infratrochlear nerves and anterior ethmoidal nerves of the user's nose.

FIG. 8 is another diagrammatic view of the nasal strip of FIGS. 3 and 4A applied to the user's nose, illustrating an epidermal traction stimulation of the infratrochlear nerves and the anterior ethmoidal nerves of the user's nose.

FIG. 9 is a diagrammatic view of an exemplary implementation of a method of using the nasal strip shown in FIGS. 1, 2A, and 2B, or the nasal strip shown in FIGS. 3, 4A, and 4B, in accordance with the inventive concepts disclosed herein.

### DETAILED DESCRIPTION OF EXEMPLARY EMBODIMENTS

The following detailed description refers to the accompanying drawings. The same reference numbers in different drawings may identify the same or similar elements.

As used herein, the terms “comprises,” “comprising,” “includes,” “including,” “has,” “having,” or any other variation thereof are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any of the following: A is true (or present) and B is false (or not present), A is false (or not present) and B is true (or present), and both A and B are true (or present).

In addition, use of the “a” or “an” is employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein, qualifiers like “substantially,” “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, and combinations thereof, for example.

The use of the term “at least one” or “one or more” will be understood to include one as well as any quantity more than one. In addition, the use of the phrase “at least one of X, V, and Z” will be understood to include X alone, V alone, and Z alone, as well as any combination of X, V, and Z.

The use of ordinal number terminology (i.e., “first,” “second,” “third,” “fourth,” etc.) is solely to differentiate between two or more items and, unless explicitly stated otherwise, is not meant to imply any sequence or order or importance to one item over another or any order of addition.

Finally, as used herein, any reference to “one embodiment” or “an embodiment” means that a particular element, feature, structure, or characteristic described in connection with the embodiment is included in at least one embodiment. The appearances of the phrase “in one embodiment” in various places in the specification do not necessarily refer to the same embodiment.

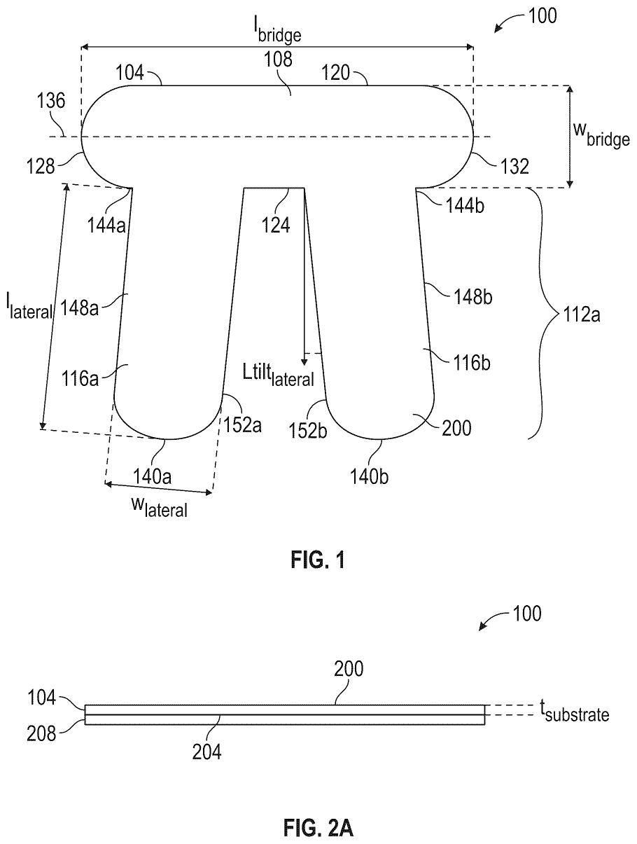

Referring now to the drawings and in particular to FIG. 1, shown therein is a top plan view of an exemplary implementation of a nasal strip 100 operable for treating obstructive sleep apnea (OSA). The nasal strip 100 generally includes a substrate 104 that comprises a bridge portion 108 and one or more lateral portion pairs 116a-n, such as a first lateral portion pair 112a as shown in FIG. 1. The first lateral portion pair 112a includes a first lateral portion 116a and a second lateral portion 116b (collectively, the “lateral portions 116a-116b”). The nasal strip 100 may be configured to be attached to a user's nose, wherein the bridge portion 108 is centered across the dorsum nasi of the user's nose and the lateral portions 116a-116b extend along a nasal sidewall of the user's nose, as will be discussed in more detail below.

In one implementation, the bridge portion 108 may have a first side 120, a second side 124 opposite the first side 120, a first end 128, a second end 132 opposite the first end 128, and a longitudinal axis 136 extending between the first end 128 and the second end 132. The bridge portion 108 may have an elongated shape along the longitudinal axis 136 with a bridge length l_{bridge} between the first end 128 and the second end 132. In some implementations, the bridge length l_{bridge} is in a range between 1 centimeter (cm) and 10 cm. In one implementation, the bridge length l_{bridge} is approximately 5 cm. The bridge portion 108 may have a narrower bridge width w_{bridge} between the first side 120 and the second side 124. In some implementations, the bridge width w_{bridge} is in a range between 0.3 cm and 3 cm. In one implementation, the bridge width w_{bridge} is approximately 1.5 cm. The bridge portion 108 is sized and shaped to extend from a first nasal sidewall of a user's nose, across a bridge of the user's nose, and to a second nasal sidewall of the user's nose. It will be understood the size and shape of the bridge portion 108 may vary to accommodate different users' nose shapes and sizes.

Each of the lateral portions 116a-116b may have a distal end 140, a proximal end 144 opposite the distal end 140, an exterior side 148, and an interior side 152 opposite the exterior side 148. That is, the first lateral portion 116a may have a first distal end 140a, a first proximal end 144a opposite the first distal end 140a, a first exterior side 148a, and a first interior side 152a opposite the first exterior side 148a. The second lateral portion 116b may have a second distal end 140b (collectively, the “distal ends 140a-140b”), a second proximal end 144b (collectively, the “proximal ends 144a-144b”) opposite the second distal end 140b, a second exterior side 148b (collectively, the “exterior sides 148a-148b”), and a second interior side 152b (collectively, the “interior sides 152a-152b”) opposite the second exterior side 148b.

In one implementation, each of the lateral portions 116a-116b may extend from the proximal ends 144a-114b, proximate to the second side 124 of the bridge portion 108, to the distal ends 140a-140b of each of the lateral portions 116a-116b having a lateral length lₗₐₜₑᵣₐₗ. In some implementations, the lateral length lₗₐₜₑᵣₐₗ is in a range between 0.4 cm and 4 cm. In one implementation, the lateral length lateral is approximately 2 cm. In one implementation, each of the lateral portions 116a-116b may be orientated to be perpendicular to the bridge portion 108. In another implementation, each of the lateral portions 116a-116b may be positioned at a slight angle or orientation from perpendicularity with the bridge portion 108, such that the distal ends 140a-140b are spaced a greater distance from one another than the proximal ends 144a-144b are spaced from one another. That is, each of the lateral portions 116a-116b may have a lateral tilt ∠tiltₗₐₜₑᵣₐₗ. In some implementations, the lateral tilt ∠tiltₗₐₜₑᵣₐₗ is in a range between 1 degree (°) and 10°. In one implementation, such as is shown in FIG. 1, for example, each of the lateral portions 116a-116b has an outward lateral tilt ∠tiltₗₐₜₑᵣₐₗ of approximately 5°. Each of the lateral portions 116a-116b is sized and shaped to be positionable over a portion of an anterior ethmoidal nerve on the first nasal sidewall and the first nasal sidewall of the user's nose when the bridge portion 108 is positioned across the bridge of the user's nose. In one implementation, the first lateral portion 116a is spaced from the second lateral portion 116b.

In one implementation, the exterior sides 148a-148b of each of the lateral portion 116a-116b extends from the second side 124 of the bridge portion 108 to the distal ends 140a-140b of the lateral portions 116a-116b and may be proximate to and offset from one of the first end 128 and the second end 132 of the bridge portion 108. By way of example, the first exterior side 148a of the first lateral portion 116a is proximate to and offset from the first end 128 of the bridge portion 108, whereas the second external side 136b of the second lateral portion 116b is proximate to and offset from the second end 132 of the bridge portion 108.

In one implementation, the interior sides 152a-152b of each of the lateral portions 116a-116b extend from the second side 124 of the bridge portion 108 to the distal ends 140a-140b of the lateral portions 116a-116b, opposite the exterior sides 148a-148b of the lateral portions 116a-116b. In the example shown in FIG. 1, the first interior side 152a of the first lateral portion 116a is proximate to and offset from the second interior side 152b of the second lateral portion 116b. Each of the lateral portions 116a-116b may have a lateral width wₗₐₜₑᵣₐₗ between the exterior side 148 and the interior side 152. In some implementations, the lateral width wₗₐₜₑᵣₐₗ is in a range between 0.2 cm and 2 cm. In one implementation, the lateral width wₗₐₜₑᵣₐₗ is approximately 1 cm.

In one implementation, the substrate 104 may be constructed of a thin, light-weight, material with elastic properties, such as kinesiology tape. In some implementations, substrate 104 may be constructed from natural materials, synthetic materials, or a combination of natural and synthetic materials. For example, the substrate 104 may be constructed from, but not limited to: cotton, silk, rayon, nylon, polyester, polyethylene, polyurethane, combinations thereof, and/or the like. In one version that may be more applicable for sleeping, the substrate 104 may be constructed from cotton/nylon or cotton/spandex blend with 94-97% cotton and the remainder being either of the two synthetics. A sports version may be substantially 100% polyester. A day version may be constructed of a transparent polyurethane. The substrate 104 may be latex-free.

In one implementation, the substrate 104 may be constructed from a woven fabric that is substantially resistant to stretching in only one test direction of two test directions specified in ASTM (American Society for Testing and Materials) D3759. In some implementations, the substrate 104 may be configured to stretch up to about 152% of a length of the bridge portion and a length of the first lateral portion and a length of the second lateral portion. In other implementations, the substrate 104 may be configured to stretch in a first direction by an amount in a range between 35% and 55%. In some such implementations, the substrate 104 may be configured to stretch in a second direction perpendicular to the first direction by an amount in a range between 35% and 55%. As a result, the substrate 104 is applied stretched greater than its normal length, it will “recoil” after being applied and therefore create a tractional force on the skin.

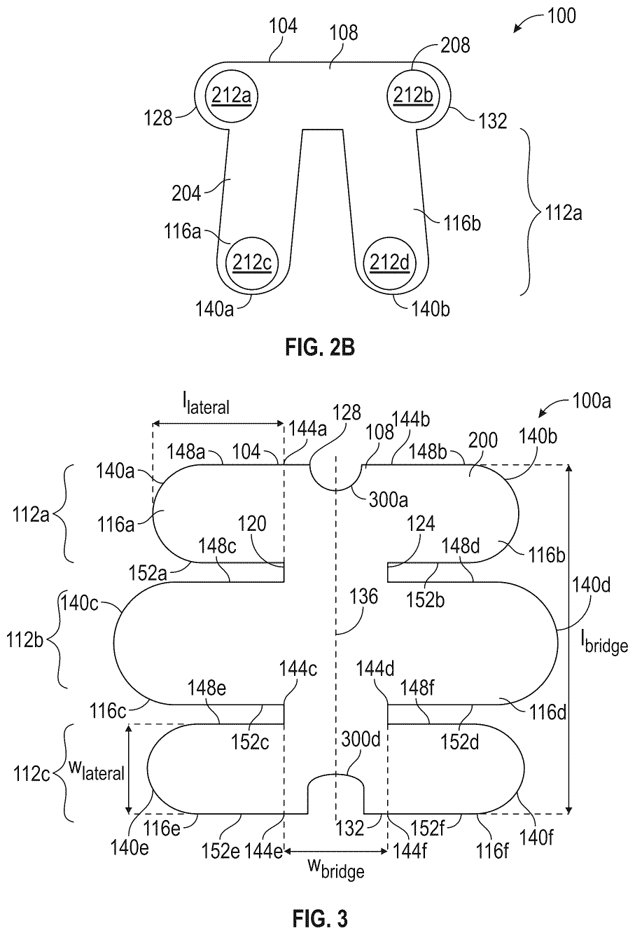

Referring now to FIG. 2A, shown therein is a side view of the nasal strip 100 shown in FIG. 1. In one implementation, the substrate 104 may further include a top surface 200 and a bottom surface 204 opposite the top surface 200. The substrate 104 may have a substrate thickness t_{substrate} extending between the top surface 200 and the bottom surface 204. In some implementations, the substrate thickness t_{substrate} measures less than approximately 30 mils.

In one implementation, the nasal strip 100 may include an adhesive layer 208 disposed on the bottom surface 204 of the substrate 104, and configured to adhere the substrate 104 to the user's nose. In some implementations, the adhesive layer 208 is disposed on the entirety of the bottom surface 204. The adhesive layer 208 may be biocompatible with the user's skin to reduce interactions between the adhesive layer 208 and the user's skin. In one implementation, the adhesive layer may be a hypoallergenic acrylic adhesive. Prior to use, the adhesive layer 208 may be covered with a removable backing (not shown).

Referring now to FIG. 2B, shown therein is a bottom plan view of the nasal strip 100 shown in FIGS. 1 and 2A. In some implementations, the adhesive layer 208 may only cover select portions of the bottom surface 204 as necessary to adhere the nasal strip 100 to the user's nose. For example, as shown in FIG. 2B, the adhesive layer 208 may include a first adhesive portion 212a proximate to the first end 128 of the bridge portion 108, a second adhesive portion 212b proximate to the second end 132 of the bridge portion 108, a third adhesive portion 212c proximate to the first distal end 140a of the first lateral portion 116a, and a fourth adhesive portion 212d proximate to the second distal end 140b of the second lateral portion 116b.

Referring now to FIG. 3, shown therein is a top plan view of another exemplary implementation of a nasal strip 100a operable for treating OSA. Like the nasal strip 100 described above, the nasal strip 100a generally includes the substrate 104 that comprises the bridge portion 108. However, unlike the nasal strip 100 described above, the nasal strip 100a includes a plurality of lateral portion pairs 112a-112n, such as the first lateral portion pair 112a, a second lateral portion pair 112b, and a third lateral portion pair 112c (collectively, the “lateral portion pairs 112a-112c”) shown in FIG. 3. Like the first lateral portion pair 112a shown in FIG. 1, the first lateral portion pair 112a shown in FIG. 3 includes the lateral portions 116a-116b (i.e., the first lateral portion 116a and the second lateral portion 116b). Further, the second lateral portion pair 112b includes a third lateral portion 116c and a fourth lateral portion 116d (collectively, the “lateral portions 116c-116d”), and the third lateral portion pair 112c includes a fifth lateral portion 116e and a sixth lateral portion 116f (collectively, the “lateral portions 116e-116f”) (the first lateral portion 116a, the second lateral portion 116b, the third lateral portion 116c, the fourth lateral portion 116d, the fifth lateral portion 116e, and the sixth lateral portion 116f, collectively, the “lateral portions 116a-116f”). The nasal strip 100a may be configured to be attached to a user's nose, wherein the bridge portion 108 is centered across the dorsum nasi of the user's nose and the lateral portions 116a-n extend along a nasal sidewall of the user's nose, as will be discussed in more detail below.

Like the bridge portion 108 shown in FIG. 1, the bridge portion 108 shown in FIG. 3 may have the first side 120, the second side 124, the first end 128, the second end 132, and the longitudinal axis 136. The bridge portion 108 is sized and shaped to extend from a first nasal sidewall of a user's nose, across a bridge of the user's nose, and to a second nasal sidewall of the user's nose.

Like the lateral portions 116a-116b shown in FIG. 1, each of the lateral portions 116a-116f shown in FIG. 3 may have the distal end 140, the proximal end 144, the exterior side 148, and the interior side 152. That is, the first lateral portion 116a may have the first distal end 140a, the first proximal end 144a, the first exterior side 148a, and the first interior side 152a; the second lateral portion 116b may have the second distal end 140b, the second proximal end 144b, the second exterior side 148b, and the second interior side 152b; the third lateral portion 116c may have a third distal end 140c, a third proximal end 144c opposite the third distal end 140c, a third exterior side 148c, and a third interior side 152c opposite the third exterior side 148c; the fourth lateral portion 116d may have a fourth distal end 140d, a fourth proximal end 144d opposite the fourth distal end 140d, a fourth exterior side 148d, and a fourth interior side 152d opposite the fourth exterior side 148d; the fifth lateral portion 116e may have a fifth distal end 140e, a fifth proximal end 144e opposite the fifth distal end 140e, a fifth exterior side 148e, and a fifth interior side 152e opposite the fifth exterior side 148e; and the sixth lateral portion 116f may have a sixth distal end 140f (collectively, the “distal ends 140a-140f”), a sixth proximal end 144f (collectively, the “proximal ends 144a-144f”) opposite the sixth distal end 140f, a sixth exterior side 148f (collectively, the “exterior sides 148a-148f”), and a sixth interior side 152f (collectively, the “interior sides 152a-152f”) opposite the sixth exterior side 148f.

Like the lateral portions 116a-116b shown in FIG. 1, each of the lateral portions 116a-116f shown in FIG. 3 may extend from the proximal ends 144a-144f, proximate to the second side 124 of the bridge portion 108, to the distal ends 140a-140f of each of the lateral portions 116a-116f. In one implementation, each of the lateral portions 116a-116f may be orientated to be perpendicular to the bridge portion 108. In another implementation, each of the lateral portions 116a-116f may be positioned at a slight angle or orientation from perpendicularity with the bridge portion 108, such that the distal ends 140a-140f are spaced a greater distance from one another than the proximal ends 144a-144f are spaced from one another. Each of the lateral portions 116a-116f is sized and shaped to be positionable over a portion of an anterior ethmoidal nerve on one of the first nasal sidewall and the second nasal sidewall of the user's nose when the bridge portion 108 is positioned across the bridge of the user's nose. In one implementation, each of the lateral portions 116a-116f is spaced a distance from at least one other one of the lateral portions 116a-116f.

Unlike the substrate 104 shown in FIG. 1, the substrate 104 in FIG. 3 may define one or more of a first notch 300a proximate to the first end 128 of the substrate 104 and a second notch 300b proximate to the second end 132 of the substrate 104.

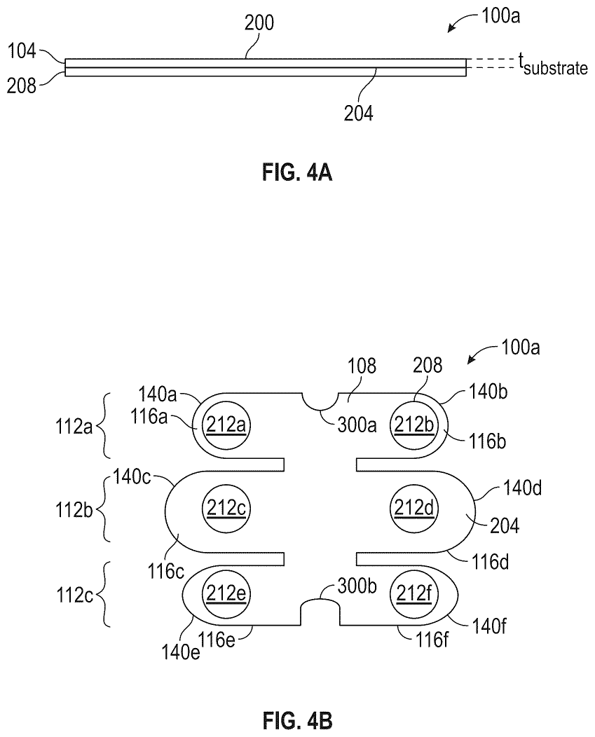

Referring now to FIG. 4A, shown therein, is a side view of the nasal strip 100a shown in FIG. 3. Like the nasal strip 100 shown in FIG. 2A, the substrate 104 may include the top surface 200, the bottom surface 204, and the adhesive layer 208 disposed on the bottom surface 204 of the substrate 104.

Referring now to FIG. 4B, shown therein, is a bottom plan view of the nasal strip 100a shown in FIGS. 3 and 4A. Like the adhesive layer 208 shown in FIG. 2B, the adhesive layer 208 in FIG. 4B may only cover select portions of the bottom surface 204 as necessary to adhere the nasal strip 100 to the user's nose. For example, as shown in FIG. 4B, the adhesive layer 208 may include the first adhesive portion 212a proximate to the first distal end 140a of the first lateral portion 116a, the second adhesive portion 212b proximate to the second distal end 140b of the second lateral portion 116b, the third adhesive portion 212c proximate to the third distal end 140c of the third lateral portion 116c, the fourth adhesive portion 212d proximate to the fourth distal end 140d of the fourth lateral portion 116d, a fifth adhesive portion 212e proximate to the fifth distal end 140e of the fifth lateral portion 116e, and a sixth adhesive portion 212f proximate to the sixth distal end 140f of the sixth lateral portion 116f.

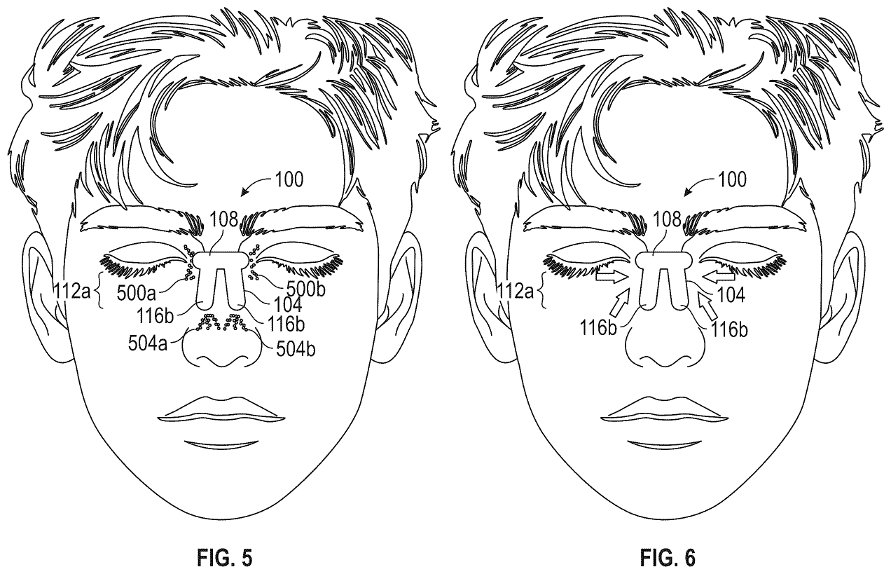

Referring now to FIG. 5, the nasal strip 100 is shown in use, positioned proximate to infratrochlear nerves 500a-500b and anterior ethmoidal nerves 504a-504b of the user's nose in accordance with the inventive concepts disclosed herein. As depicted, the bridge portion 108 may be longitudinally centered on the dorsum nasi of the user's nose just below an imaginary center point between a user's pupils. The first end 128 of the bridge portion 108 and the second end 132 of the bridge portion 108 may be disposed on opposing nasal sidewalls of the user's nose. For example, in FIG. 5, the first end 128 is shown attached to the user's right nasal sidewall, and the second end 132 is shown attached to the user's left nasal sidewall. The lateral portions 116 may extend from the bridge portion 108 along the nasal sidewall and dorsum nasi of the user's nose. For example, the first lateral portion 116a is positioned on the user's nose, such that the first exterior side 148a of the first lateral portion 116a is disposed along the nasal sidewall on the right side of the user's nose. The first interior side 152a of the first lateral portion 116a is disposed along the right side of the dorsum nasi of the user's nose. Similarly, the second lateral portion 116b is positioned on the user's nose, such that the second exterior side 148b of the second lateral portion 116b is disposed along the nasal sidewall on the left side of the user's nose. The second interior side 152b of the second lateral portion 116b is disposed along the left side of the dorsum nasi of the user's nose.

The infratrochlear nerves 500a-500b are a small branch of the ophthalmic division of the trigeminal nerve extending along the orbit of the eye and traveling anteriorly toward the medial aspect of the nasal bridge. The anterior ethmoidal nerves 504a-504b are also branches of the ophthalmic division of the trigeminal nerve, which enter the nasal cavity through the ethmoidal foramen and branch out to the anterior and upper regions of the nasal cavity. The infratrochlear nerves 500a-500b and the anterior ethmoidal nerves 504a-504b are subcutaneous nerves; however, in FIG. 5, the infratrochlear nerves 500a-500b and the anterior ethmoidal nerves 504a-504b are shown superimposed over the nasal strip 100 and the user's nose to demonstrate the relationship between the infratrochlear nerves 500a-500b and the anterior ethmoidal nerves 504a-504b and the nasal strip 100 when attached to the user's nose. As shown in FIG. 5, the bridge portion 108 may be configured to align with at least a portion of the infratrochlear nerves 500a-500b, and the lateral portions 116 may be configured to align with at least a portion of the anterior ethmoidal nerves 504a-504b.

Turning now to FIG. 6, the nasal strip 100 shown in FIGS. 1, 2A, and 2B is shown in use as applied to the user's nose to generate an epidermal tractional force. In one implementation, the bridge portion 108 may be configured to be longitudinally stretched by the user, providing an outward force on the first end 128 and an opposing outward force on the second end 132. While maintaining the bridge portion 108 in a stretched configuration, the bridge portion 108 may be applied, such that the adhesive layer 208 disposed on the bottom surface 204 affixes the bridge portion 108 to the user's nose in the stretched configuration. As applied to the user's nose in the stretched configuration, the bridge portion 108 may provide a tractional force parallel to a surface of application in a direction of the bridge of the user's nose to stimulate the infratrochlear nerves 500a-500b, as indicated by the arrows.

In one implementation, the lateral portions 116 may be configured to be affixed to the user's nose in a stretched configuration to provide a tractional force to stimulate the anterior ethmoidal nerves 504a-504b. In one implementation, the user would first attach the bridge portion 108, as described above, and then provide a longitudinal force on the distal ends 140a-140b of the lateral portions 116 away from the bridge portion 108 to stretch the lateral portions 116 along the nasal sidewall before being attached to the user's nose. The adhesive layer 208 disposed on the bottom surface 204 may be used to maintain the lateral portions 116 in a stretched configuration after being applied to the user's nose. The lateral portions 116 of the substrate 104, held in the stretched configuration, may be operable to provide a tractional force parallel to the surface of application in a direction of the bridge to stimulate the anterior ethmoidal nerves 504a-504b. The tractional force is depicted by the arrows along each nasal sidewall of the user's nose.

In one implementation, the combined tractional force provided by the stretched bridge portion 108 and the stretched lateral portions 116 instigate and maintain a gentle, concerted stimulation of the infratrochlear and anterior ethmoidal nerves. The stimulation engenders neuroconnectivity and sensation, which positively engages an activation energy of target impulses and reflexes, resulting in neuro-harmonically affected impulses and reflexes that cause key tissue of the oral cavity and pharynx to rise and expand. Expanding these key tissues provides a necessary histological rigidity and flexibility, improves impulse conductivity, and improves structural histological interfaces of the otolaryngological area through neuro-harmonic connective innervation stimulation. The improved histological conductivity of the otolaryngological area aids in preventing and ceasing OSA.

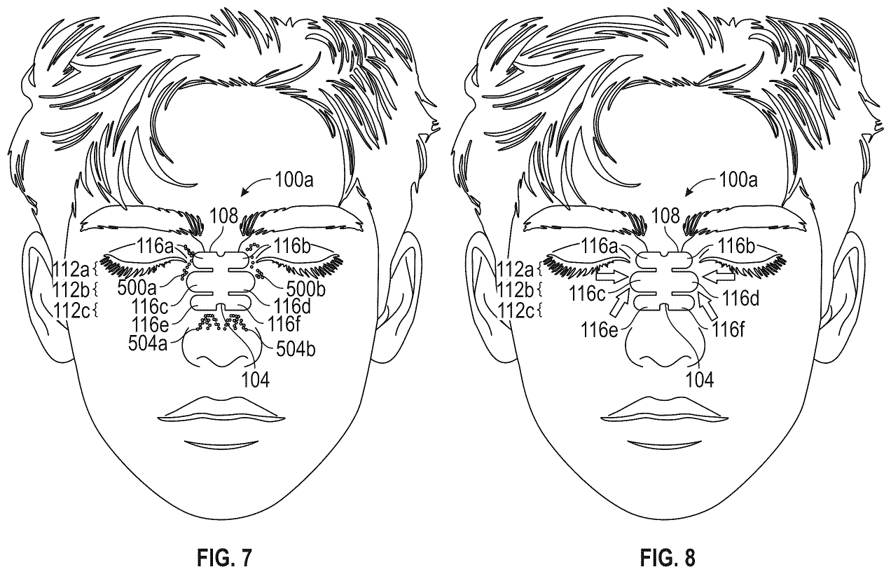

Referring now to FIG. 7, the nasal strip 100a of FIGS. 3, 4A, and 4B is shown in use, positioned proximate to infratrochlear nerves 500a-500b and anterior ethmoidal nerves 504a-504b of the user's nose in accordance with the present disclosure. As depicted, the bridge portion 108 may be latitudinally centered on the dorsum nasi of the user's nose just below an imaginary center point between a user's pupils. The first side 120 of the bridge portion 108 and the second side 124 of the bridge portion 108 may be disposed on opposing nasal sidewalls of the user's nose. For example, in FIG. 7, the first side 120 is shown attached to the user's right nasal sidewall, and the second side 124 is shown attached to the user's left nasal sidewall. The lateral portions 116 may extend from the bridge portion 108 across the nasal sidewalls and away from the dorsum nasi of the user's nose. For example, the first lateral portion 116a is positioned over the user's nose such that the first distal end 140a extends across the nasal sidewalls on the right side of the user's nose and away from the dorsum nasi of the user's nose and the second lateral portion 116b is positioned over the user's nose such that the second distal end 140b extends across nasal sidewalls on the left side of the user's nose and away from the dorsum nasi of the user's nose.

Turning now to FIG. 8, the nasal strip 100a of FIGS. 3, 4A, and 4B is shown in use as applied to the user's nose to generate an epidermal tractional force. In one implementation, the bridge portion 108 may be configured to be latitudinally stretched by the user, providing an outward force on the first side 120 and an opposing outward force on the second side 124. While maintaining the bridge portion 108 in a stretched configuration, the bridge portion 108 may be applied, such that the adhesive layer 208 disposed on the bottom surface 204 affixes the bridge portion 108 to the user's nose in the stretched configuration. As applied to the user's nose in the stretched configuration, the bridge portion 108 may provide a tractional force parallel to a surface of application in a direction of the bridge of the user's nose to stimulate the infratrochlear nerves 500a-500b, as indicated by the arrows.

In one implementation, the lateral portions 116 may be configured to be affixed to the user's nose in a stretched configuration to provide a tractional force to stimulate the anterior ethmoidal nerves 504a-504b. In one implementation, the user would first attach the bridge portion 108, as described above, and then provide a longitudinal force on the distal ends 140a-140f of the lateral portions 116 away from the bridge portion 108 to stretch the lateral portions 116 across the nasal sidewall before being attached to the user's nose. The adhesive layer 208 disposed on the bottom surface 204 may be used to maintain the lateral portions 116 in a stretched configuration after being applied to the user's nose. The lateral portions 116 of the substrate 104, held in the stretched configuration, may be operable to provide a tractional force parallel to the surface of application in a direction of the bridge to stimulate the anterior ethmoidal nerves 504a-504b. The tractional force is depicted by the arrows along each nasal sidewall of the user's nose.

In one implementation, the combined tractional force provided by the stretched bridge portion 108 and the stretched lateral portions 116 instigate and maintain a gentle, concerted stimulation of the infratrochlear and anterior ethmoidal nerves. The stimulation engenders neuroconnectivity and sensation, which positively engages an activation energy of target impulses and reflexes, resulting in neuro-harmonically affected impulses and reflexes that cause key tissue of the oral cavity and pharynx to rise and expand. Expanding these key tissues provides a necessary histological rigidity and flexibility, improves impulse conductivity, and improves structural histological interfaces of the otolaryngological area through neuro-harmonic connective innervation stimulation. The improved histological conductivity of the otolaryngological area aids in preventing and ceasing OSA.

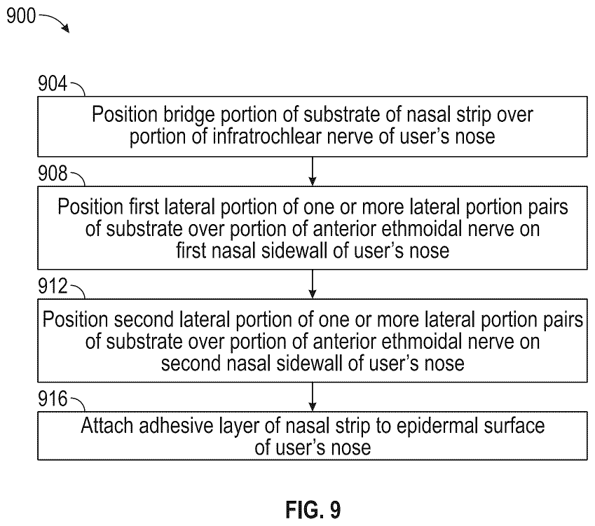

Referring now to FIG. 9, shown therein is an exemplary implementation of a method 900 of applying the nasal strip 100 shown in FIGS. 1, 2A, and 2B, or the nasal strip 100a shown in FIGS. 3, 4A, and 4B, in accordance with the present disclosure. As shown in FIG. 9, the method 900 generally comprises the steps of: positioning a bridge portion 108 of a substrate 104 of a nasal strip 100, 100a over at least a portion of an infratrochlear nerve 500a-500b of a user's nose (step 904); positioning a first lateral portion 116a of one or more lateral portion pairs 112a-112n of the substrate 104 over a portion of an anterior ethmoidal nerve 504a-504b on the first nasal sidewall of the user's nose (step 908); positioning a second lateral portion 116b of the one or more lateral portion pairs 112a-112n of the substrate 104 over a portion of an anterior ethmoidal nerve 504a-504b on the second nasal sidewall of the user's nose (step 912); and attaching an adhesive layer 208 of the nasal strip 100, 100a to an epidermal surface of the user's nose (step 916).

In some implementations, the method 900 further comprises the step of, prior to positioning the first lateral portion 116a of the one or more lateral portion pairs 112a-112n of the substrate 104 over the portion of the anterior ethmoidal nerve 504a-504b on the first nasal sidewall of the user's nose, applying a longitudinal force to a first distal end 140a of the first lateral portion 116a in a direction away from the bridge portion 108 to stretch the first lateral portion 116a along or across the first nasal sidewall of the user's nose.

In some implementations, the method 900 further comprises the step of, prior to positioning the second lateral portion 116b of the one or more lateral portion pairs 112a-112n of the substrate 104 over the portion of the anterior ethmoidal nerve 504a-504b on the second nasal sidewall of the user's nose, applying a longitudinal force to a second distal end 140b of the second lateral portion 116b in a direction away from the bridge portion 108 to stretch the second lateral portion 116b along or across the second nasal sidewall of the user's nose.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A nasal strip for treating obstructive sleep apnea (OSA), comprising:
a substrate formed of a flexible, elastic material and having a skin-facing surface and a non-skin-facing surface opposite the skin-facing surface, the substrate having:
a bridge portion having a first end, a second end opposite the first end, a first side, a second side opposite the first side, and a longitudinal axis extending between the first end and the second end, the bridge portion being sized and dimensioned to extend between a first nasal sidewall of a user's nose and a second nasal sidewall of the user's nose opposite the first nasal sidewall and across a bridge of the user's nose and to be positionable over at least a portion of an infratrochlear nerve of the user's nose to provide an epidermal tractional force parallel to a surface of application on the user's nose and in a direction toward the bridge of the user's nose to stimulate the infratrochlear nerves of the user's nose;
a first lateral portion extending from the second side of the bridge portion, the first lateral portion being sized and dimensioned to be positionable over a portion of an anterior ethmoidal nerve on the first nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the first nasal sidewall of the user's nose;
a second lateral portion extending from the second side of the bridge portion, the second lateral portion being sized and dimensioned to be positionable over a portion of an anterior ethmoidal nerve on the second nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the second nasal sidewall of the user's nose; and
an adhesive layer disposed on the skin-facing surface of the substrate, the adhesive layer being operable to attach the substrate to an epidermal surface of the user's nose.

2. The nasal strip of claim 1, wherein the first lateral portion and the second lateral portion are spaced a distance from one another.

3. The nasal strip of claim 2, wherein the first lateral portion has a first proximal end and a first distal end opposite the first proximal end and the second lateral portion has a second proximal end and a second distal end opposite the second proximal end, the first lateral portion and the second lateral portion being angled relative to the bridge portion such that the first distal end and the second distal end are spaced a greater distance from one another than the first proximal end and the second proximal end are spaced from one another.

4. The nasal strip of claim 1, wherein the bridge portion has a bridge length extending between the first end and the second end, the bridge length being in a range between 1 cm and 10 cm.

5. The nasal strip of claim 1, wherein the bridge portion has a bridge width extending between the first side and the second side, the bridge width being in a range between 0.3 cm and 3 cm.

6. The nasal strip of claim 1, wherein the first lateral portion has a first proximal end, a first distal end opposite the first proximal end, and a first lateral length extending between the first proximal end and the first distal end, and the second lateral portion has a second proximal end, a second distal end opposite the second proximal end, and a second lateral length extending between the second proximal end and the second distal end, at least one of the first lateral length and the second lateral length being in a range between 0.4 cm and 4 cm.

7. The nasal strip of claim 1, wherein the first lateral portion has a first exterior side, a first interior side opposite the first exterior side, and a first lateral width extending between the first exterior side and the first interior side, and the second lateral portion has a second exterior side, a second interior side opposite the second exterior side, and a second lateral width extending between the second exterior side and the second interior side, at least one of the first lateral width and the second lateral width being in a range between 0.2 cm and 2 cm.

8. The nasal strip of claim 1, wherein the substrate is a woven fabric configured to stretch in a first direction by an amount in a range between 35% and 55%.

9. The nasal strip of claim 1, wherein the substrate is further configured to stretch in a second direction perpendicular to the first direction by an amount in a range between 35% and 55%.

10. A nasal strip for treating obstructive sleep apnea (OSA), comprising:
a substrate formed of a flexible, elastic material and having a skin-facing surface and a non-skin-facing surface opposite the skin-facing surface, the substrate having:
a bridge portion having a first end, a second end opposite the first end, a first side, a second side opposite the first side, and a longitudinal axis extending between the first end and the second end, the bridge portion being sized and dimensioned to extend between a first nasal sidewall of a user's nose and a second nasal sidewall of the user's nose opposite the first nasal sidewall and across a bridge of the user's nose and to be positionable over at least a portion of an infratrochlear nerve of the user's nose to provide an epidermal tractional force parallel to a surface of application on the user's nose and in a direction toward the bridge of the user's nose to stimulate the infratrochlear nerves of the user's nose;
a plurality of lateral portion pairs, each of the plurality of lateral portion pairs including a first lateral portion extending from the first side of the bridge portion and a second lateral portion extending from the second side of the bridge portion; and
an adhesive layer disposed on the skin-facing surface of the substrate, the adhesive layer being operable to attach the substrate to an epidermal surface of the user's nose,
wherein the first lateral portion of each of the plurality of lateral portion pairs is sized and dimensioned to be positionable over a portion of an anterior ethmoidal nerve on the first nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the first nasal sidewall of the user's nose, and
wherein the second lateral portion of each of the plurality of lateral portion pairs is sized and dimensioned to be positionable over a portion of an anterior ethmoidal nerve on the second nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the second nasal sidewall of the user's nose.

11. The nasal strip of claim 10, wherein the first lateral portion of each of the plurality of lateral portion pairs is spaced a distance from the first lateral portion of at least one other lateral portion pair of the plurality of lateral portion pairs and the second lateral portion of each of the plurality of lateral portion pairs is spaced a distance from the second lateral portion of at least one other lateral portion pair of the plurality of lateral portion pairs.

12. The nasal strip of claim 10, wherein the bridge portion has a bridge length extending between the first end and the first end, the bridge length being in a range between 1 cm and 10 cm.

13. The nasal strip of claim 10, wherein the bridge portion has a bridge width extending between the first side and the first side, the bridge width being in a range between 0.3 cm and 3 cm.

14. The nasal strip of claim 10, wherein the first lateral portion of each of the plurality of lateral portion pairs has a first proximal end, a first distal end opposite the first proximal end, and a first lateral length extending between the first proximal end and the first distal end, and the second lateral portion has a second proximal end, a second distal end opposite the second proximal end, and a second lateral length extending between the second proximal end and the second distal end, at least one of the first lateral length and the second lateral length being in a range between 0.4 cm and 4 cm.

15. The nasal strip of claim 10, wherein the first lateral portion of each of the plurality of lateral portion pairs has a first exterior side, a first interior side opposite the first exterior side, and a first lateral width extending between the first interior side and the first exterior side, and the second lateral portion of each of the plurality of lateral portion pairs has a second exterior side, a second interior side opposite the second exterior side, and a second lateral width extending between the second exterior side and the second interior side, at least one of the first lateral width and the second lateral width being in a range between 0.2 cm and 2 cm.

16. The nasal strip of claim 10, wherein the substrate is a woven fabric configured to stretch in a first direction by an amount in a range between 35% and 55%.

17. The nasal strip of claim 16, wherein the substrate is further configured to stretch in a second direction perpendicular to the first direction by an amount in a range between 35% and 55%.

18. A method of applying a nasal strip for treating obstructive sleep apnea (OSA), comprising:
positioning a bridge portion of a substrate of the nasal strip over at least a portion of an infratrochlear nerve of a user's nose, the substrate being formed of a flexible, elastic material and having a skin-facing surface and a non-skin-facing surface opposite the skin-facing surface, the bridge portion having a first end, a second end opposite the first end, a first side, a second side opposite the first side, and a longitudinal axis extending between the first end and the second end, the bridge portion being sized and dimensioned to extend between a first nasal sidewall of the user's nose and a second nasal sidewall of the user's nose opposite the first nasal sidewall and across a bridge of the user's nose to provide an epidermal tractional force parallel to a surface of application on the user's nose and in a direction toward the bridge of the user's nose to stimulate the infratrochlear nerves of the user's nose;
positioning a first lateral portion of one or more lateral portion pairs of the substrate over a portion of an anterior ethmoidal nerve on the first nasal sidewall of the user's nose, the first lateral portion of each of the one or more lateral portion pairs being sized and dimensioned to be positionable over a portion of an anterior ethmoidal nerve on the first nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the first nasal sidewall of the user's nose;
positioning a second lateral portion of the one or more lateral portion pairs of the substrate over a portion of an anterior ethmoidal nerve on the second nasal sidewall of the user's nose, the second lateral portion of each of the one or more lateral portion pairs being sized and dimensioned to be positionable over a portion of an anterior ethmoidal nerve on the second nasal sidewall of the user's nose when the bridge portion is positioned across the bridge of the user's nose to provide an epidermal tractional force parallel to the surface of application on the user's nose and in a direction toward the bridge portion to stimulate the anterior ethmoidal nerve on the second nasal sidewall of the user's nose; and
attaching an adhesive layer of the nasal strip to an epidermal surface of the user's nose, the adhesive layer being disposed on the skin-facing surface of the substrate.

19. The method of claim 18, wherein the first lateral portion of each of the one or more lateral portion pairs has a first proximal end and a first distal end opposite the first proximal end, the method further comprising, prior to positioning the first lateral portion of the one or more lateral portion pairs of the substrate over the portion of the anterior ethmoidal nerve on the first nasal sidewall of the user's nose, applying a longitudinal force to the first distal end of the first lateral portion in a direction away from the bridge portion to stretch the first lateral portion along or across the first nasal sidewall of the user's nose.

20. The method of claim 18, wherein the second lateral portion of each of the one or more lateral portion pairs has a second proximal end and a second distal end opposite the second proximal end, the method further comprising, prior to positioning the second lateral portion of the one or more lateral portion pairs of the substrate over the portion of the anterior ethmoidal nerve on the second nasal sidewall of the user's nose, applying a longitudinal force to the second distal end of the second lateral portion in a direction away from the bridge portion to stretch the second lateral portion along or across the second nasal sidewall of the user's nose.
